(19) United States
(12) Patent Application Publication
(10) Pub. No.: US 2022/0095958 A1
(43) Pub. Date: Mar. 31, 2022

(54) MEASUREMENT APPARATUS, DETERMINATION METHOD, AND NON-TRANSITORY RECORDING MEDIUM

(71) Applicant: CASIO COMPUTER CO., LTD., Tokyo (JP)

(72) Inventors: Katsunori ISHII, Tokyo (JP); Hiroyasu IDE, Tokyo (JP)

(73) Assignee: CASIO COMPUTER CO., LTD., Tokyo (JP)

(21) Appl. No.: 17/486,294

(22) Filed: Sep. 27, 2021

(30) Foreign Application Priority Data
Sep. 30, 2020 (JP) 2020-165107

(51) Int. Cl. A61B 5/11; A61B 5/00; G01P 7/00; G01P 1/12

(52) U.S. Cl. CPC A61B 5/1123; A61B 5/6802; G01P 1/127; A61B 5/6838; G01P 7/00; A61B 5/6823

(57) ABSTRACT

A measurement apparatus is equipped with a processor. The processor acquires first velocity data, which is data on a velocity in a first direction, and second velocity data, which is data on a velocity in a second direction orthogonal to the first direction, on the basis of accelerations acquired at an acceleration sensor. The processor calculates a first coefficient on the basis of the acquired first velocity data and the acquired second velocity data. The processor determines whether the measurement apparatus is installed in normal orientation on the basis of the calculated first coefficient.

### CROSS-REFERENCE TO RELATED APPLICATION

This application is based upon and claims the benefit of priority under 35 USC 119 of Japanese Patent Application No. 2020-165107 filed on Sep. 30, 2020, the entire disclosure of which, including the description, claims, drawings, and abstract, is incorporated herein by reference in its entirety.

### FIELD

This application relates to a measurement apparatus, a determination method, and a non-transitory recording medium.

### BACKGROUND

International Publication No. WO 2016/024565 discloses a motion capture device installed at a periodic exercise site and including a case that accommodates a three-dimensional acceleration sensor and an arithmetic processing section, for example.

### SUMMARY

A measurement apparatus according to a first aspect of the disclosure includes a processor. The processor acquires first velocity data and second velocity data on the basis of accelerations acquired at an acceleration sensor. The first velocity data is data on a velocity in a first direction, and the second velocity data is data on a velocity in a second direction orthogonal to the first direction. The processor calculates a first coefficient on the basis of the acquired first velocity data and the acquired second velocity data. The processor determines whether the measurement apparatus is installed in normal orientation on the basis of the calculated first coefficient.

A measurement apparatus according to a second aspect of the disclosure includes a processor. The processor acquires velocity data on the basis of an acceleration acquired at an acceleration sensor, and acquires angular velocity data from an angular velocity sensor for measuring an angular velocity. The velocity data is data on a velocity in a certain direction. The processor calculates a coefficient on the basis of the acquired velocity data and the acquired angular velocity data. The processor determines whether the measurement apparatus is installed in normal orientation on the basis of the calculated coefficient.

A determination method according to a third aspect of the disclosure, involves: acquiring first velocity data and second velocity data on the basis of accelerations acquired at an acceleration sensor, the first velocity data being data on a velocity in a first direction, the second velocity data being data on a velocity in a second direction orthogonal to the first direction; calculating a first coefficient on the basis of the acquired first velocity data and the acquired second velocity data; and determining whether the measurement apparatus is installed in normal orientation on the basis of the calculated first coefficient.

A non-transitory recording medium according to a fourth aspect of the disclosure stores a program, which causes a computer to: acquire first velocity data and second velocity data on the basis of accelerations acquired at an acceleration sensor, the first velocity data being data on a velocity in a first direction, the second velocity data being data on a velocity in a second direction orthogonal to the first direction; calculate a first coefficient on the basis of the acquired first velocity data and the acquired second velocity data; and determine whether the measurement apparatus is installed in normal orientation on the basis of the calculated first coefficient.

### BRIEF DESCRIPTION OF THE DRAWINGS

A more complete understanding of this application can be obtained when the following detailed description is considered in conjunction with the following drawings, in which:

FIG. 1A is a front view illustrating an appearance of a measurement apparatus according to Embodiment 1;

FIG. 1B is a side view illustrating an appearance of the measurement apparatus according to Embodiment 1;

FIG. 2 illustrates the measurement apparatus according to Embodiment 1 worn by a user;

FIG. 3 is a block diagram illustrating a configuration of the measurement apparatus according to Embodiment 1;

FIG. 4 is a flowchart of a determination process executed at a processing unit of the measurement apparatus according to Embodiment 1;

FIG. 5 illustrates a velocity in the Y direction and a velocity in the Z direction during running of the user wearing the measurement apparatus in the inside-out orientation in Embodiment 1;

FIG. 6 illustrates a velocity in the Y direction and a velocity in the Z direction during running of the user wearing the measurement apparatus in the normal orientation in Embodiment 1;

FIG. 7 is a block diagram illustrating a configuration of a measurement apparatus according to Embodiment 2;

FIG. 8 is a flowchart of a determination process executed at a processing unit of the measurement apparatus according to Embodiment 2;

FIG. 9 illustrates a velocity in the X direction and an angular velocity about the Z axis during running of the user wearing the measurement apparatus in the inside-out orientation in Embodiment 2; and

FIG. 10 illustrates a velocity in the X direction and an angular velocity about the Z axis during running of the user wearing the measurement apparatus in the normal orientation in Embodiment 2.

### DETAILED DESCRIPTION

### Embodiment 1

A measurement apparatus 1 according to Embodiment 1 is described below with reference to the accompanying drawings. In the drawings, the components identical or corresponding to each other are provided with the same reference symbol. The measurement apparatus 1 according to Embodiment 1 is worn by a user to measure information on movement of the user.

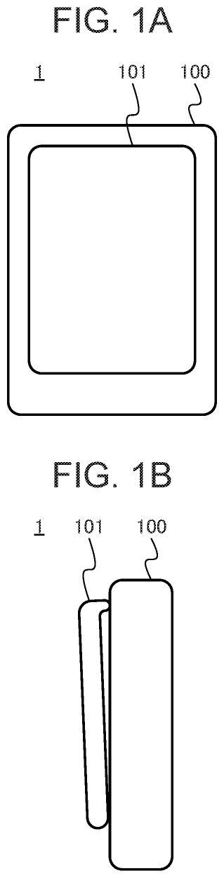

FIG. 1A is a front view illustrating an appearance of the measurement apparatus 1. FIG. 1B is a side view illustrating an appearance of the measurement apparatus 1. As illustrated in FIGS. 1A and 1B, the measurement apparatus 1 is equipped with a housing 100 and a clip 101.

The housing 100 is a case that accommodates a sensor unit 200, a storage unit 300, a communication unit 400, and a processing unit 500, which are described below. The housing 100 may be made of a metal or resin, for example, but these examples are not intended to limit the scope of the disclosure.

The clip 101 is bonded to one surface of the housing 100. The clip 101 is urged toward the housing 100 and tightly holds an object between the housing 100 and the clip 101. The clip 101 may be made of a metal or resin, for example, and may further contain an elastic member for urging the clip 101, but these examples are not intended to limit the scope of the disclosure.

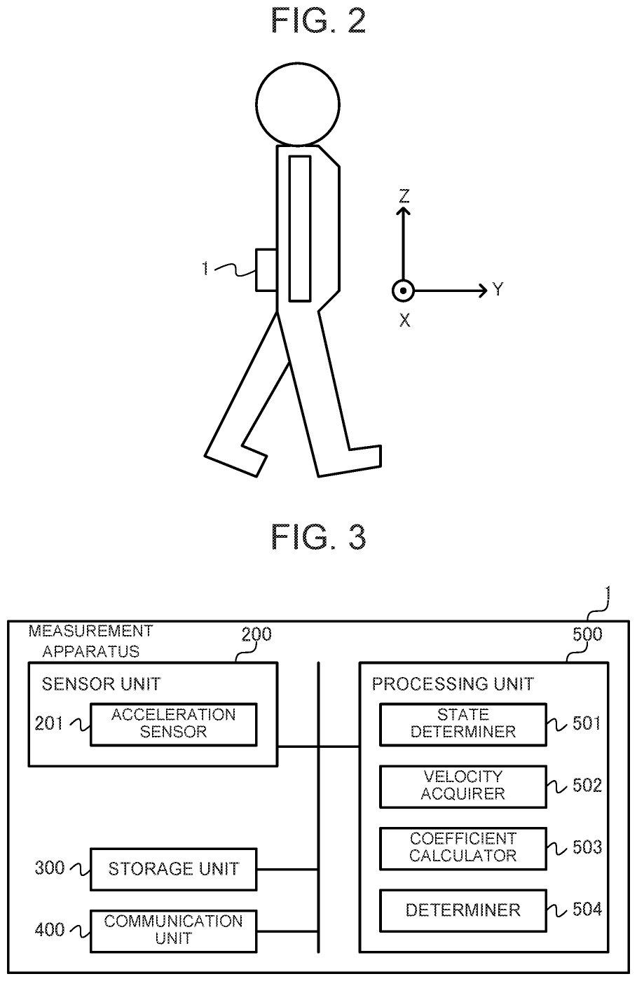

FIG. 2 illustrates the measurement apparatus 1 worn by the user. As illustrated in FIG. 2, the measurement apparatus 1 is installed at a position on the rear side of the waist of the user. The housing 100 and the clip 101 tightly hold pants or a belt therebetween, for example, and thereby install the measurement apparatus 1 on the user. The measurement apparatus 1 is installed such that the surface opposite to the surface provided with the clip 101 is in contact with the body of the user. As illustrated in FIG. 2, the traveling direction of the user is defined as the positive direction in the Y axis while the vertically upward direction is defined as the positive direction in the Z axis.

The surface of the measurement apparatus 1 opposite to the surface provided with the clip 101 needs to be in contact with the body of the user and is hereinafter referred to as “contact surface”. The installation of the measurement apparatus 1 on the body of the user with the contact surface in contact with the body is hereinafter referred to as “proper installation”. In contrast, the installation of the measurement apparatus 1 on the body of the user with the surface opposite to the contact surface in contact with the body is hereinafter referred to as “reverse installation”.

FIG. 3 is a block diagram illustrating a configuration of the measurement apparatus 1 according to Embodiment 1. As illustrated in FIG. 3, the measurement apparatus 1 includes the sensor unit 200, the storage unit 300, the communication unit 400, and the processing unit 500.

The sensor unit 200 includes an acceleration sensor 201 to measure accelerations. Examples of the acceleration sensor 201 may include a semiconductor acceleration sensor and piezoelectric acceleration sensor, but these examples are not intended to limit the scope of the disclosure.

The storage unit 300 stores programs executed at the processing unit 500, data calculated at the processing unit 500, and data measured at the sensor unit 200. Examples of the storage unit 300 may include a random access memory (RAM), flash memory, read only memory (ROM), erasable programmable ROM (EPROM), and electrically erasable programmable ROM (EEPROM), but these examples are not intended to limit the scope of the disclosure.

The communication unit 400 is a communication interface to transmit and receive signals to and from the outside of the measurement apparatus 1. Examples of the communication unit 400 may include a wireless communication interface and wired communication interface, but these examples are not intended to limit the scope of the disclosure.

The processing unit 500 includes a state determiner 501, a velocity acquirer 502, a coefficient calculator 503, and a determiner 504. Examples of the processing unit 500 may include a central processing unit (CPU), but this example is not intended to limit the scope of the disclosure. The processing unit 500 is also called a processor 500.

The state determiner 501 acquires acceleration data in the X, Y, and Z directions from the acceleration sensor 201. On the basis of the acceleration data, the state determiner 501 determines whether the user wearing the measurement apparatus 1 is in a state of running or in another state. The state of running is hereinafter referred to as “running state”. The state determiner 501 executes the determination, for example, by a procedure of a support vector machine, but this example is not intended to limit the scope of the disclosure.

The velocity acquirer 502 acquires acceleration data in the Y and Z directions from the acceleration sensor 201, and integrates the individual values of the acceleration data to acquire velocity data in the Y and Z directions.

The coefficient calculator 503 acquires the velocity data in the Y direction and the velocity data in the Z direction acquired at the velocity acquirer 502, and calculates a correlation coefficient (first coefficient) between the acquired velocity data in the Y direction and the acquired velocity data in the Z direction.

If the absolute value of the calculated correlation coefficient is equal to or higher than a threshold (second threshold), the coefficient calculator 503 adds the calculated correlation coefficient to the value of an evaluation counter, and causes the resulting value to be stored into the storage unit 300. In contrast, when the absolute value of the calculated correlation coefficient is lower than the threshold, the coefficient calculator 503 does not add the correlation coefficient to the value of the evaluation counter.

The determiner 504 determines whether the measurement apparatus 1 is installed in the normal orientation. Specifically, the determiner 504 acquires the value of the evaluation counter from the storage unit 300. When the value of the evaluation counter is higher than a positive threshold (first threshold), the determiner 504 determines that the measurement apparatus 1 is installed in the inside-out orientation, that is, determines reverse installation. When the value of the evaluation counter is lower than a negative threshold (fourth threshold), the determiner 504 determines that the measurement apparatus 1 is installed in the normal orientation.

The determiner 504 resets the value of the evaluation counter stored in the storage unit 300 at the start of the determination process.

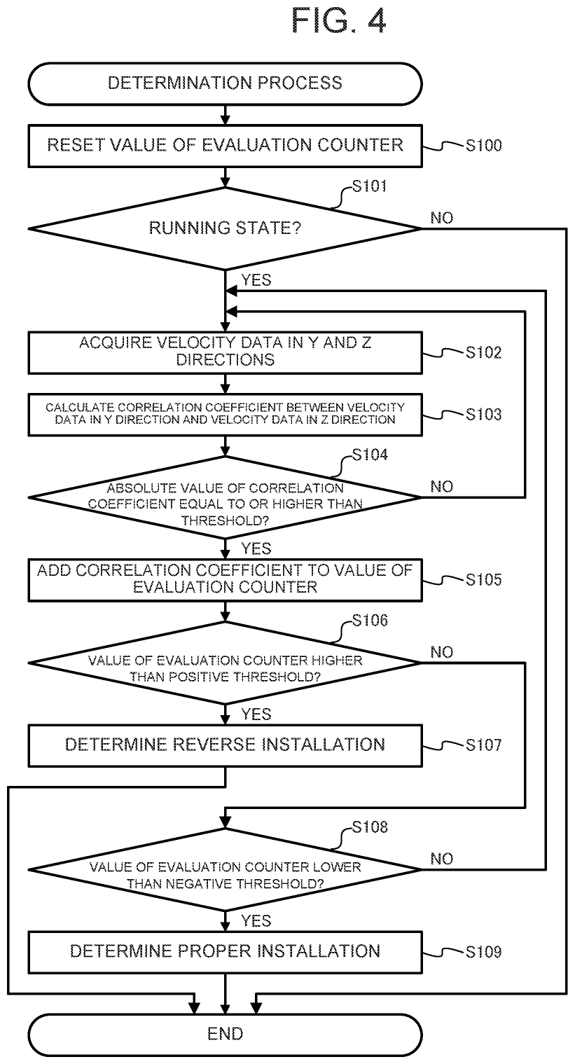

FIG. 4 is a flowchart of the determination process executed at the processing unit 500 of the measurement apparatus 1 according to Embodiment 1. The determination process is described below with reference to the flowchart of FIG. 4.

At the start of the determination process, the determiner 504 resets the value of the evaluation counter stored in the storage unit 300 (Step S100).

After reset of the value of the evaluation counter, the state determiner 501 acquires acceleration data in the X, Y, and Z directions from the acceleration sensor 201, and determines whether the user wearing the measurement apparatus 1 is in a running state on the basis of the acceleration data (Step S101). When the user is determined to be not in a running state (Step S101: NO), the determination process is terminated.

In contrast, when the user is determined to be in a running state (Step S101: YES), the velocity acquirer 502 acquires acceleration data in the Y and Z directions from the acceleration sensor 201, and integrates the individual values of the acceleration data to acquire velocity data in the Y and Z directions (Step S102).

After acquisition of the velocity data at the velocity acquirer 502, the coefficient calculator 503 acquires the velocity data in the Y direction and the velocity data in the Z direction acquired at the velocity acquirer 502, and calculates a correlation coefficient between the acquired velocity data in the Y direction and the acquired velocity data in the Z direction (Step S103).

After calculation of the correlation coefficient at the coefficient calculator 503, the coefficient calculator 503 determines whether the absolute value of the calculated correlation coefficient is equal to or higher than the threshold (Step S104). When the correlation coefficient is determined to be lower than the threshold (Step S104: NO), the process returns to Step S102.

In contrast, when determining the correlation coefficient to be equal to or higher than the threshold (Step S104: YES), the coefficient calculator 503 adds the calculated correlation coefficient to the value of the evaluation counter, and causes the resulting value to be stored into the storage unit 300 (Step S105).

After addition of the correlation coefficient to the value of the evaluation counter at the coefficient calculator 503, the determiner 504 acquires the value of the evaluation counter from the storage unit 300, and determines whether the value of the evaluation counter is higher than the positive threshold (Step S106).

If determining the value of the evaluation counter to be higher than the positive threshold (Step S106: YES), the determiner 504 determines that the measurement apparatus 1 is installed in the inside-out orientation (Step S107), followed by termination of the determination process.

In contrast, when determining the value of the evaluation counter to be equal to or lower than the positive threshold (Step S106: NO), the determiner 504 acquires the value of the evaluation counter from the storage unit 300, and determines whether the value of the evaluation counter is lower than the negative threshold (Step S108).

If determining the value of the evaluation counter to be lower than the negative threshold (Step S108: YES), the determiner 504 determines that the measurement apparatus 1 is installed in the normal orientation (Step S109), followed by termination of the determination process.

In contrast, when the value of the evaluation counter is determined to be equal to or higher than the negative threshold (Step S108: NO), the process returns to Step S102.

The measurement apparatus 1 according to Embodiment 1 includes the above-described configuration and executes the determination process, and can thus prevent measurement to be continued despite of installation of the measurement apparatus 1 in the inside-out orientation. The measurement apparatus 1 determines the inside-out orientation, and can thus avoid impairment of the accuracy of measured data and results of analysis of the data and avoid output of incorrect results of analysis due to installation in the inside-out orientation.

The coefficient calculator 503 of the measurement apparatus 1 determines whether the absolute value of the correlation coefficient is equal to or higher than the threshold, and adds the correlation coefficient to the value of the evaluation counter when the correlation coefficient is equal to or higher than the threshold. The measurement apparatus 1 can thus determine the orientation on the basis of only data having a size sufficient for determination of the orientation, leading to improvement of the accuracy of determination of the orientation.

The measurement apparatus 1 determines that the measurement apparatus 1 is installed in the inside-out orientation when the determiner 504 determines the value of the evaluation counter to be higher than the positive threshold. The measurement apparatus 1 can thus determine the orientation after collection of the number of data sufficient for determination of the orientation, and avoid incorrect determination.

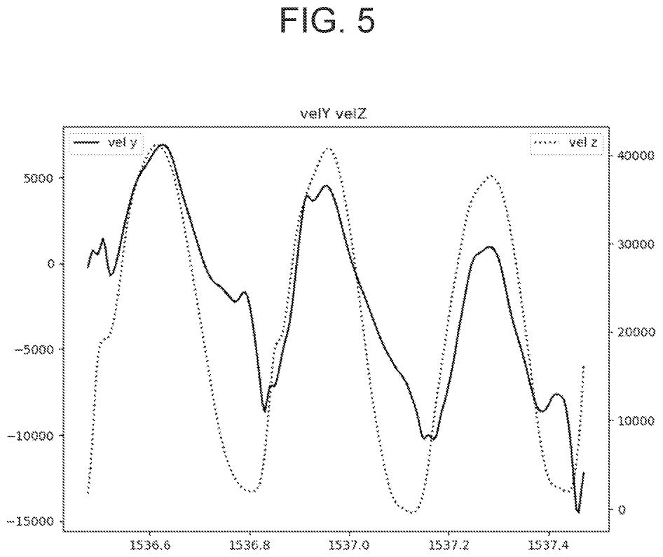

FIG. 5 illustrates a velocity in the Y direction and a velocity in the Z direction during running of the user wearing the measurement apparatus 1 in the inside-out orientation. As illustrated in FIG. 5, during running of the user wearing the measurement apparatus 1 in the inside-out orientation, the velocity in the Y direction (velY: solid line) tends to have the same phase as that of the velocity in the Z direction (velZ: dotted line). The calculation of a correlation coefficient in this state provides a positive correlation coefficient. In view of the above-described tendency of movement of the user, the measurement apparatus 1 determines that the measurement apparatus 1 is installed in the inside-out orientation when the determiner 504 determines the value of the evaluation counter to be higher than the positive threshold, thereby achieving accurate determination of the orientation.

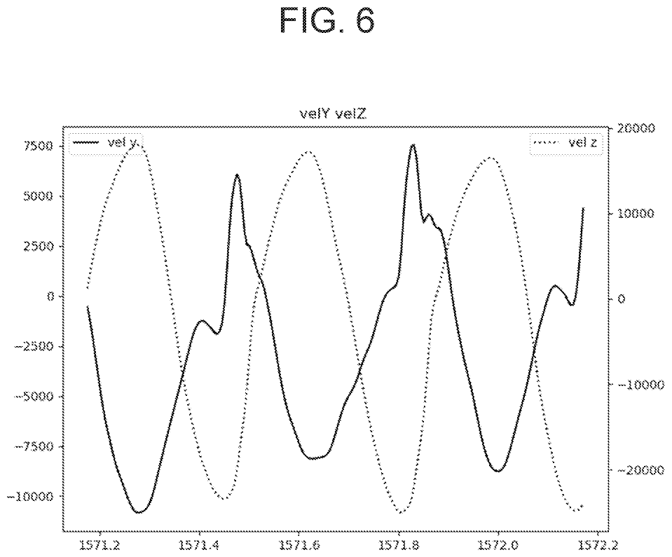

FIG. 6 illustrates a velocity in the Y direction and a velocity in the Z direction during running of the user wearing the measurement apparatus 1 in the normal orientation. As illustrated in FIG. 6, during running of the user wearing the measurement apparatus 1 in the normal orientation, the velocity in the Y direction (velY: solid line) tends to have the opposite phase to that of the velocity in the Z direction (velZ: dotted line). The calculation of a correlation coefficient in this state provides a negative correlation coefficient. In view of the above-described tendency of movement of the user, the measurement apparatus 1 determines that the measurement apparatus 1 is installed in the normal orientation when the determiner 504 determines the value of the evaluation counter to be lower than the negative threshold, thereby achieving accurate determination of the orientation.

### Embodiment 2

The measurement apparatus 1 according to Embodiment 2 is described below with reference to the accompanying drawings. In the drawings, the components identical or corresponding to each other are provided with the same reference symbol.

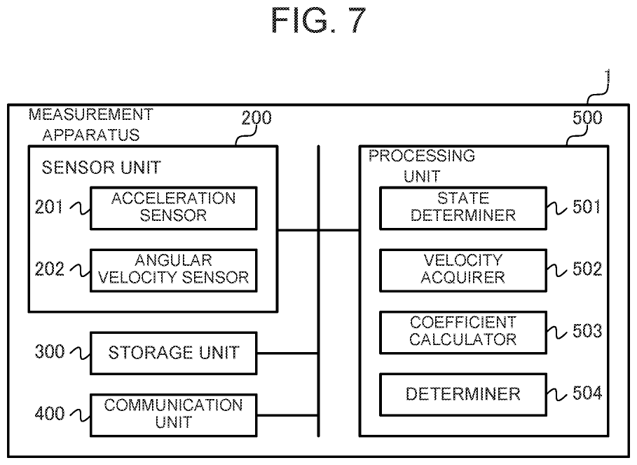

FIG. 7 is a block diagram illustrating a configuration of the measurement apparatus 1 according to Embodiment 2. As illustrated in FIG. 7, the sensor unit 200 of the measurement apparatus 1 according to Embodiment 2 includes an angular velocity sensor 202.

The sensor unit 200 includes the angular velocity sensor 202 to measure an angular velocity. Examples of the angular velocity sensor 202 may include a gyro sensor, but this example is not intended to limit the scope of the disclosure.

The state determiner 501 acquires acceleration data in the X, Y, and Z directions from the acceleration sensor 201, and determines whether the user wearing the measurement apparatus 1 is in a state of walking or running, or in another state, on the basis of the acceleration data. The states of walking and running are collectively referred to as “moving states”. The state determiner 501 executes the determination, for example, by a procedure of a support vector machine, but this example is not intended to limit the scope of the disclosure.

The velocity acquirer 502 acquires acceleration data in the X direction from the acceleration sensor 201, and integrates the value of the acceleration data to acquire velocity data in the X direction. The velocity acquirer 502 also acquires angular velocity data about the Z axis from the angular velocity sensor 202.

The coefficient calculator 503 acquires the velocity data in the X direction and the angular velocity data about the Z axis acquired at the velocity acquirer 502, and calculates a correlation coefficient (second coefficient) between the velocity data in the X direction and the angular velocity data about the Z axis.

If the absolute value of the calculated correlation coefficient is equal to or higher than a threshold (third threshold), the coefficient calculator 503 adds the calculated correlation coefficient to the value of the evaluation counter, and causes the resulting value to be stored into the storage unit 300. In contrast, when the absolute value of the calculated correlation coefficient is lower than the threshold, the coefficient calculator 503 does not add the correlation coefficient to the value of the evaluation counter.

The determiner 504 acquires the value of the evaluation counter from the storage unit 300. When the value of the evaluation counter is higher than the positive threshold, the determiner 504 determines that the measurement apparatus 1 is installed in the inside-out orientation. When the value of the evaluation counter is lower than the negative threshold, the determiner 504 determines that the measurement apparatus 1 is installed in the normal orientation.

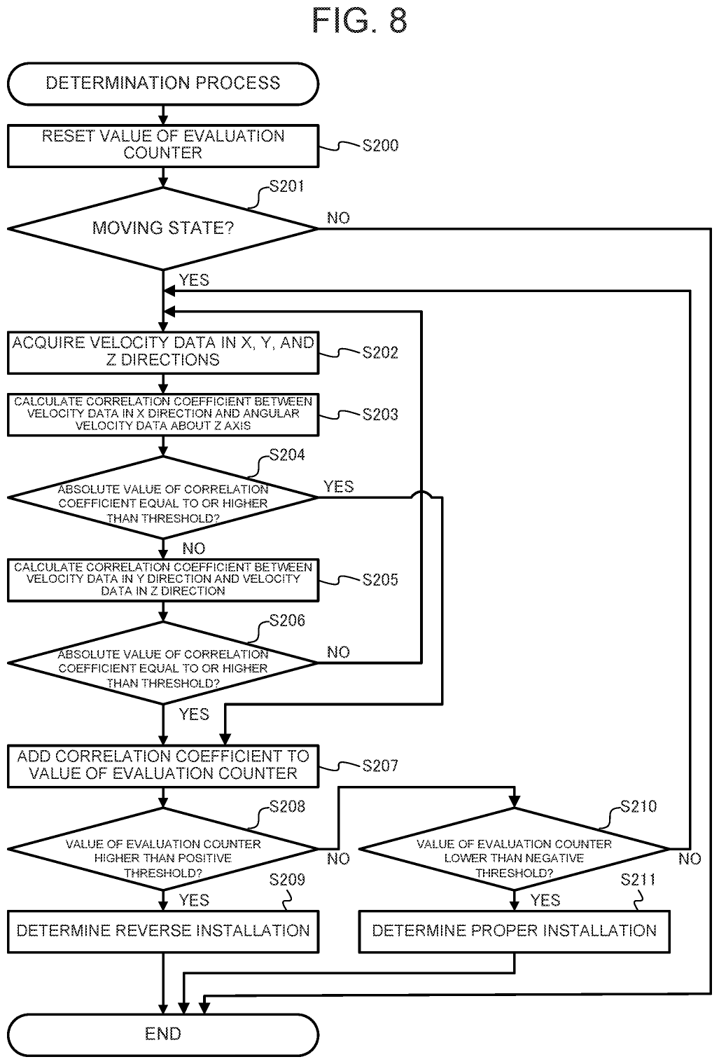

FIG. 8 is a flowchart of a determination process executed at the processing unit 500 of the measurement apparatus 1 according to Embodiment 2. The determination process is described below with reference to the flowchart of FIG. 8.

At the start of the determination process, the determiner 504 resets the value of the evaluation counter stored in the storage unit 300 (Step S200).

After reset of the value of the evaluation counter, the state determiner 501 acquires acceleration data in the X, Y, and Z directions from the acceleration sensor 201, and determines whether the user wearing the measurement apparatus 1 is in a moving state on the basis of the acceleration data (Step S201). When the user is determined to be not in a moving state (Step S201: NO), the determination process is terminated.

In contrast, when the user is determined to be in a moving state (Step S201: YES), the velocity acquirer 502 acquires acceleration data in the X, Y, and Z directions from the acceleration sensor 201 and acquires angular velocity data about the Z axis from the angular velocity sensor 202, and then integrates the individual values of the acceleration data to acquire velocity data in the X, Y, and Z directions (Step S202).

After acquisition of the velocity data at the velocity acquirer 502, the coefficient calculator 503 acquires the velocity data in the X direction and the angular velocity data about the Z axis acquired at the velocity acquirer 502, and calculates a correlation coefficient between the velocity data in the X direction and the angular velocity data about the Z axis (Step S203).

After calculation of the correlation coefficient at the coefficient calculator 503, the coefficient calculator 503 determines whether the absolute value of the calculated correlation coefficient is equal to or higher than the threshold (Step S204). When the correlation coefficient is determined to be equal to or higher than the threshold (Step S204: YES), the process proceeds to Step S207 described below.

In contrast, when the correlation coefficient is determined to be lower than the threshold (Step S204: NO), the coefficient calculator 503 acquires the velocity data in the Y direction and the velocity data in the Z direction acquired at the velocity acquirer 502, and calculates a correlation coefficient between the acquired velocity data in the Y direction and acquired the velocity data in the Z direction (Step S205).

After calculation of the correlation coefficient at the coefficient calculator 503, the coefficient calculator 503 determines whether the absolute value of the calculated correlation coefficient is equal to or higher than the threshold (Step S206). When the correlation coefficient is determined to be lower than the threshold (Step S206: NO), the process returns to Step S202.

In contrast, when the absolute value of either of the correlation coefficients calculated at the coefficient calculator 503 is determined to be equal to or higher than the threshold (Step S204: YES, or Step S206: YES), the coefficient calculator 503 adds the calculated correlation coefficient to the value of the evaluation counter, and causes the resulting value to be stored into the storage unit 300 (Step S207).

After addition of the correlation coefficient to the value of the evaluation counter at the coefficient calculator 503, the determiner 504 acquires the value of the evaluation counter from the storage unit 300, and determines whether the value of the evaluation counter is higher than the positive threshold (Step S208).

If determining the value of the evaluation counter to be higher than the positive threshold (Step S208: YES), the determiner 504 determines that the measurement apparatus 1 is installed in the inside-out orientation (Step S209), followed by termination of the determination process.

In contrast, when determining the value of the evaluation counter to be equal to or lower than the positive threshold (Step S208: NO), the determiner 504 acquires the value of the evaluation counter from the storage unit 300, and determines whether the value of the evaluation counter is lower than the negative threshold (Step S210).

If determining the value of the evaluation counter to be lower than the negative threshold (Step S210: YES), the determiner 504 determines that the measurement apparatus 1 is installed in the normal orientation (Step S211), followed by termination of the determination process.

In contrast, when the value of the evaluation counter is determined to be equal to or higher than the negative threshold (Step S210: NO), the process returns to Step S202.

The measurement apparatus 1 according to Embodiment 2 includes the above-described configuration and executes the determination process, and can thus bring about the same advantageous effects as the measurement apparatus 1 according to Embodiment 1.

The coefficient calculator 503 of the measurement apparatus 1 determines whether the absolute value of the correlation coefficient between the velocity data in the X direction and the angular velocity data about the Z axis is equal to or higher than the threshold. When the correlation coefficient is equal to or higher than the threshold, the coefficient calculator 503 adds the correlation coefficient to the value of the evaluation counter. When the correlation coefficient is lower than the threshold, the coefficient calculator 503 proceeds to calculation of a correlation coefficient between the velocity data in the Y direction and the velocity data in the Z direction. The measurement apparatus 1 can thus apply a significantly high correlation coefficient to determination of the orientation, leading to improvement of the accuracy of determination of the orientation. Specifically, the correlation coefficient between the velocity data in the X direction and the angular velocity data about the Z axis tends to be significantly high during running of the user, and tends to be significantly low during walking of the user. The use of this correlation coefficient between the velocity data in the Y direction and the velocity data in the Z direction can thus contribute to improvement of the accuracy of determination of the orientation.

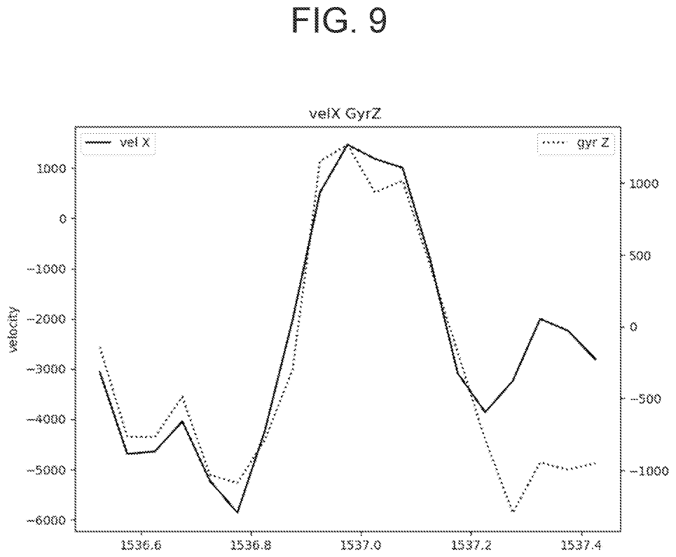

FIG. 9 illustrates a velocity in the X direction and an angular velocity about the Z axis during running of the user wearing the measurement apparatus 1 in the inside-out orientation. As illustrated in FIG. 9, during running of the user wearing the measurement apparatus 1 in the inside-out orientation, the velocity in the X direction (velX: solid line) tends to have the same phase as that of the angular velocity about the Z axis (gyrZ: dotted line). The calculation of a correlation coefficient in this state provides a positive correlation coefficient. In view of the above-described tendency of movement of the user, the measurement apparatus 1 determines that the measurement apparatus 1 is installed in the inside-out orientation when the determiner 504 determines the value of the evaluation counter to be higher than the positive threshold, thereby achieving accurate determination of the orientation.

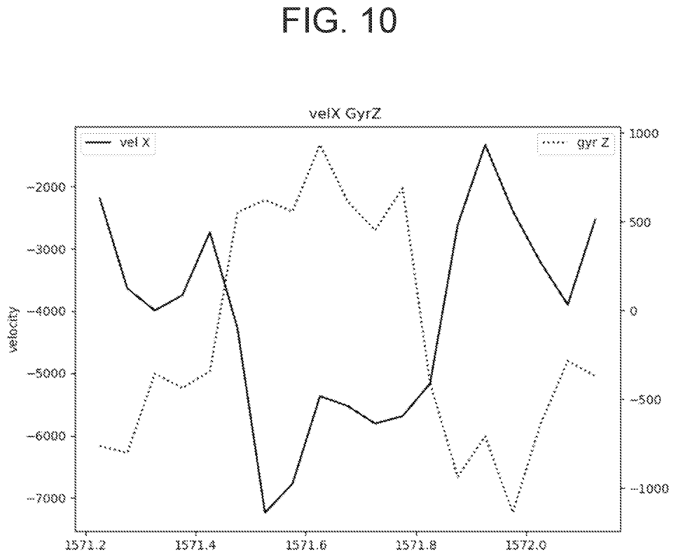

FIG. 10 illustrates a velocity in the X direction and an angular velocity about the Z axis during running of the user wearing the measurement apparatus 1 in the normal orientation. As illustrated in FIG. 10, during running of the user wearing the measurement apparatus 1 in the normal orientation, the velocity in the X direction (velX: solid line) tends to have the opposite phase to that of the angular velocity about the Z axis (gyrZ: dotted line). The calculation of a correlation coefficient in this state provides a negative correlation coefficient. In view of the above-described tendency of movement of the user, the measurement apparatus 1 determines that the measurement apparatus 1 is installed in the normal orientation when the determiner 504 determines the value of the evaluation counter to be lower than the negative threshold, thereby achieving accurate determination of the orientation.

(Modification)

The above-described embodiments of the disclosure are mere examples and not to be construed as limiting the application scope of the disclosure. That is, the embodiments of the disclosure may be provided with various modifications, and any modified embodiment can be encompassed in the scope of the disclosure.

Although the measurement apparatus 1 includes the sensor unit 200 in the above-described embodiments, this configuration is a mere example. The measurement apparatus 1 may acquire data in real time from a separate sensor apparatus, and execute a determination process for determining the orientation of the sensor apparatus. In this case, the measurement apparatus 1 may be disposed at any site provided that the measurement apparatus 1 is coupled to the sensor apparatus.

Although the sensor unit 200 includes the acceleration sensor 201 to measure accelerations and the angular velocity sensor 202 to measure an angular velocity in the above-described embodiments, this configuration is a mere example. The sensor unit 200 may include a geomagnetic sensor to measure geomagnetism.

Although the coefficient calculator 503 calculates the correlation coefficient between the velocity data in the Y direction and the velocity data in the Z direction, or the correlation coefficient between the velocity data in the X direction and the angular velocity data about the Z axis in the above-described embodiments, this configuration is a mere example. The coefficient calculator 503 may calculate a coefficient on the basis of the phases of velocity data and another velocity data, or the phases of velocity data and angular velocity data.

Although the determiner 504 determines reverse installation of the measurement apparatus 1 when the value of the evaluation counter is higher than the positive threshold and determines proper installation of the measurement apparatus 1 when the value of the evaluation counter is lower than the negative threshold in the above-described embodiments, this configuration is a mere example. The determiner 504 may determine proper installation or reverse installation of the measurement apparatus 1 on the basis of the first threshold, instead of the value of the evaluation counter.

Although the measurement apparatus 1 includes the communication unit 400 as a communication interface to transmit and receive signals to and from the outside of the measurement apparatus 1 in the above-described embodiments, this configuration is a mere example. The measurement apparatus 1 may include an interface, which a non-transitory recording medium can be attached to and detached from, cause the non-transitory recording medium to store data, and provide the data to an external apparatus via the non-transitory recording medium coupled to the external apparatus.

Although the velocity acquirer 502 acquires velocity data when the state determiner 501 determines a running state of the user in Embodiment 1, this configuration is a mere example. The velocity acquirer 502 may acquire velocity data when the state determiner 501 determines that the user wearing the measurement apparatus 1 is in a walking state. In this case, the coefficient calculator 503 preferably adds the calculated correlation coefficient of which the sign is inverted to the value of the evaluation counter.

Although the measurement apparatus 1 determines reverse installation by calculating the correlation coefficient between the velocity data in the X direction and the angular velocity data about the Z axis and calculating the correlation coefficient between the velocity data in the Y direction and the velocity data in the Z direction in Embodiment 2, this configuration is a mere example. The measurement apparatus 1 may determine reverse installation only by calculating the correlation coefficient between the velocity data in the X direction and the angular velocity data about the Z axis, without calculating the correlation coefficient between the velocity data in the Y direction and the velocity data in the Z direction.

As well as a measurement apparatus preliminarily having a configuration for performing the functions according to the disclosure, a program may be applied to cause an existing measurement apparatus to function as the measurement apparatus according to the disclosure. That is, a program for achieving the functions of the measurement apparatus illustrated in the embodiments and the modification may be applied so as to be executable at the CPU for controlling the existing measurement apparatus, for example, and thereby cause the existing measurement apparatus to function as the measurement apparatus according to the disclosure. In addition, a determination method according to the disclosure can be executed using the measurement apparatus.

Such a program can be applied in any procedure. For example, the program may be stored in a non-transitory computer-readable recording medium, such as flexible disk, compact disc ROM (CD-ROM), digital versatile disc ROM (DVD-ROM), or memory card to be applied. Alternatively, the program may be superimposed on a carrier wave and applied via a communication medium, such as the Internet. For example, the program may be posted on a bulletin board system (BBS) on a communication network to be distributed. The program may be activated and executed under the control of an operation system (OS), like other application programs, thereby achieving the above-described process.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. A measurement apparatus comprising:
a processor, wherein
the processor
acquires first velocity data and second velocity data on basis of accelerations acquired at an acceleration sensor, the first velocity data being data on a velocity in a first direction, the second velocity data being data on a velocity in a second direction orthogonal to the first direction,
calculates a first coefficient on basis of the acquired first velocity data and the acquired second velocity data, and
determines whether the measurement apparatus is installed in normal orientation on basis of the calculated first coefficient.

2. The measurement apparatus according to claim 1, wherein
the processor
adds the calculated first coefficient to a value of an evaluation counter, and
determines whether the measurement apparatus is installed in the normal orientation on basis of the value of the evaluation counter to which the calculated first coefficient is added and a predetermined fourth threshold.

3. The measurement apparatus according to claim 2, wherein the processor adds the calculated first coefficient to the value of the evaluation counter when an absolute value of the calculated first coefficient is higher than a second threshold.

4. The measurement apparatus according to claim 1, wherein
the processor
acquires third velocity data on basis of an acceleration acquired at the acceleration sensor, and acquires angular velocity data from an angular velocity sensor for measuring an angular velocity, the third velocity data being data on a velocity in a third direction orthogonal to the first and second directions,
calculates a second coefficient on basis of the acquired angular velocity data and the acquired third velocity data, and
determines whether the measurement apparatus is installed in the normal orientation on basis of the calculated second coefficient and the calculated first coefficient.

5. The measurement apparatus according to claim 4, wherein
the processor
adds the calculated first coefficient and the calculated second coefficient to a value of an evaluation counter, and
determines whether the measurement apparatus is installed in the normal orientation on basis of the value of the evaluation counter to which the calculated first coefficient is added and a predetermined fourth threshold.

6. The measurement apparatus according to claim 4, wherein the processor adds the calculated second coefficient to a value of an evaluation counter when an absolute value of the calculated second coefficient is higher than a third threshold.

7. The measurement apparatus according to claim 5, wherein the processor adds the calculated second coefficient to the value of the evaluation counter when an absolute value of the calculated second coefficient is higher than a third threshold.

8. The measurement apparatus according to claim 6, wherein the processor calculates the first coefficient when the absolute value of the calculated second coefficient is lower than the third threshold.

9. The measurement apparatus according to claim 7, wherein the processor calculates the first coefficient when the absolute value of the calculated second coefficient is lower than the third threshold.

10. The measurement apparatus according to claim 1, wherein
the processor
determines whether a user is in a running state on basis of data related to an acquired velocity, and
calculates the first coefficient when the processor determines that the user is in the running state.

11. The measurement apparatus according to claim 4, wherein
the processor
determines whether a user is in at least one of a walking state and a running state on basis of data related to an acquired velocity, and
calculates the second coefficient when the processor determines that the user is in at least one of the walking state and the running state.

12. The measurement apparatus according to claim 1, wherein the processor determines whether the measurement apparatus is in reverse installation on basis of the calculated first coefficient.

13. The measurement apparatus according to claim 12, wherein the measurement apparatus in the reverse installation indicates that the measurement apparatus is installed on a body of a user such that the body is in contact with a part of the measurement apparatus opposite to a part intended to be in contact with the body.

14. A measurement apparatus comprising a processor, wherein
the processor
acquires velocity data on basis of an acceleration acquired at an acceleration sensor, and acquires angular velocity data from an angular velocity sensor for measuring an angular velocity, the velocity data being data on a velocity in a certain direction,
calculates a coefficient on basis of the acquired velocity data and the acquired angular velocity data, and
determines whether the measurement apparatus is installed in normal orientation on basis of the calculated coefficient.

15. A determination method comprising:
acquiring first velocity data and second velocity data on basis of accelerations acquired at an acceleration sensor, the first velocity data being data on a velocity in a first direction, the second velocity data being data on a velocity in a second direction orthogonal to the first direction;
calculating a first coefficient on basis of the acquired first velocity data and the acquired second velocity data; and
determining whether the measurement apparatus is installed in normal orientation on basis of the calculated first coefficient.

16. A non-transitory recording medium storing a program, the program causing a computer to:
acquire first velocity data and second velocity data on basis of accelerations acquired at an acceleration sensor, the first velocity data being data on a velocity in a first direction, the second velocity data being data on a velocity in a second direction orthogonal to the first direction;
calculate a first coefficient on basis of the acquired first velocity data and the acquired second velocity data; and
determine whether the measurement apparatus is installed in normal orientation on basis of the calculated first coefficient.
